SEQUENCE LISTING (16 sequences)

SEQ ID NO: 1 - DNA, 642 bp, Populus trichocarpa
atggatatgattgagcaatctgcagcaatatctccgatcaatgttccatctcaattctcctactctccatcctcttcttcaccaccttcttctcatcattcttctccttcaaagtcttctccaaatcttactccaccttcagctgccactccccctcctcctccgccagttgttagcccttgtgctgcctgcaaaatccttcgccggcgttgcgttgacaagtgtgttttagctccttattttcctccttctgagccatacaagttcaccattgctcatagggtgtttggagccagcaacatcatcaagtttttgcaggaactgccagagtcccagagagcagatgcagtgagcagcatggtttatgaagccaatgcgaggatcagagacccggtttatggctgtgctggtgcaatttctcagcttcaaaaacaagttagtgacctccaagcacagctagccaaggcacaagctgaggtggtgaacatgcaatgccagcaagccaatttagttgctttactctgcatggaaatgacacagtctcaacaagaacccatcttgcaacaacaccaatacgttgacacaagctgctttcttgacgagaacaacttgggcacatcatgggaacctctttggacatga

SEQ ID NO: 2 - DNA, 696 bp, Populus trichocarpa
atgcggatgagttgtaatggttgcagaatccttcgcaagggctgtggtgataattgtagtataaaaccttgccttcaatggattgaaactcccgattcccaagccaatgccaccctcttccttgctaaattctacggtcgtgctggactcatgaacctcatcaatgcttgccctcagcatctccgtccagatacgtttaagtccttattatacgaggcatgtgggcggattgtgaatccagtttcgggatcagtcgggttgttaagcaccgggtcatggcagcaatgtcaagccgccgtggaagccgttctgaaaggcgagcccatcactcaaattgcatcaactgatcagcttacactaggcggctctgatacgcgccacgtgtcaagagaggaggactggtctgcatctgatcagccccgtaaaatcaagtccaagcgtcaattcaatcgatcaacttcaaagcggaagccgagcaggactgaggctgagcacacatgtttcgagttcatgattggatttgatggcttgcggtcagtgagtcctgactctgtgttgagttggcgtccgagtttggggtctgacaatgaggaaactgacagtatagggtctgtggagactgtagaggcttctaagcttgatgagccagccgaaggaagtgatttggatttagacttgaccttgggtcattattga

SEQ ID NO: 3 - DNA, 441 bp, Populus trichocarpa
atgaaggagagtggtcgaaaacttggtgcactttcgccatgcgcagcatgcaagcttcttaggaggagatgtgctcaagactgtatgtttgctccttactttccagctgacgagcctcagaagtttgccagtgtgcacaaggtctttggtgctagcaatgtcaacaaaatgttacaggaattacctgagcaccaacgaagtgatgcagtaagttccatggtgtatgaagcaaatgcaagggttcgtgacccagtttatggttgtgttggtgcaatatcatctttgcagaaacaaattgattcactacaaacccagttggcaattgcacaagctgaggtagtgcacatgagagtgcggcaattcacttcttcttccaatccaggagtcatggacatggcagttgatcaggccaccatgggagagtctctgtggtcatgctag

SEQ ID NO: 4 - DNA, 702 bp, Populus trichocarpa
atgcggatgagttgtaatggatgtagagttttacgcaaaggctgtagtgaaaactgtagtattaggccttgtttacagtggatcaagagttctgagtctcaagccaacgctactgtttttcttgccaagttttatggccgtgctggccttatgaacctcatcaacgctggccctgaacatctccgtcctgcgatttttaggtcgttgctctacgaagcatgcgggaggatagtgaacccgatttatggctcggttgggctgatgtggtcgggaagctggcagctttgtcaagccgctgttgaagccgtactaaaaggtgctccgataactccaattaactctgaagctgcagttaatggccatggccccccgctaaaagtttatgatatacgacacgtttcgaaagaagaaaactcggctgcgtcaaatgatgcaaaccgagccaggactcggtgccggtcttcggtgagtcaccaatccgagttggcagtgttggatggcgacagcaaagagtctgatgagagcatggcccatgacgttgcgagtaacggaatttctgggcttgaacttggcctggacttgaccctgggccttgagcccgtgtcacgtgcacatcacgtggttcccgtgaagaagagaagggtggaagcttacgggtctggtgatgttgacacgtgtaagatggagctcagacttgaataa

SEQ ID NO: 5 - DNA, 741 bp, Populus trichocarpa
atgagtaccgcaactacaaaccctataaatactggtgctggtggtggaagtagtggcggtggaggtggtggtggcggcggtggtggcggtggtggtgggccatgcggtgcttgtaaattcttgaggaggaagtgtgtgccagggtgtatatttgcaccttattttgactcagagcaaggtgcagcacattttgcagcggtgcataaggtttttggtgcaagtaacgtgtcaaagcttttattacacattcctgtccataaaagacttgatgctgtggtcacaatttgttatgaggctcaagctcgcttaagagatccagtctatggctgtgttgctcacatctttgctcttcaacaacaggtagtaaatttacaagcagaactctcatacttgcaagcccatctagcagcattggaggttccgtcgccacctcctccgcccccgccaacactagtgaccccgcctccactctcaatagcagacctaccatcagcctcttcaattcccgccgcgtacgatttatcatgtctttttgatccaatgatgcaaccctcatggtccaacatgcagccgcagcggcaaatggatatccgtcagtttggaggcagcggtggctcatcaggaactggtggcggtgatctccaagcattggcgcgtgaactccttcatagacgaggatctccaccacccgggttcatgtcatgtagtgacacattagcatcaccatctatctctaaatga

SEQ ID NO: 6 - DNA, 672 bp, Populus trichocarpa
atgtccagagacagggagagatctgaagagttaggaaagaggatcaagagggagagtgatgcatcttcttttatggggaggagacaaatgttaggtcctcctgtaattttgaacactgttacaccttgtgctgcatgtaaacttttaagaagaagatgtgctgaagagtgccccttttctccatatttttctccacatgaaccccagaaatttgctgctgtccacaaagtctatggtgcaagcaatgtctccaagctgctaatggaggtgccagaaagtcaaagagttgatactgcaaatagtctagtttatgaagcaaacttgaggctaagagatccagtatatggctccatgggtgcaatttcagctttacaacaacaaattcaatcattacaagctgaacttagtgcaataagggctgagatactcaactacaaatacagagaagctgctgctgctactaacatcatttcttctactcatcctgctttagtttcttctgcgaccgtgtccatttcaacaccatcacaaacgcttgctccaccaccacaaccacctcctccttcagttgttgtttcttcatcttcctcttcttctctttatacaccaccaactagcacatcaggttatagcactatttcaagtgaaaataatgtcccatattttgattaa

SEQ ID NO: 7 - DNA, 492 bp, Populus trichocarpa
atgagaaatcaggagcctcgtgcaagctcttcatgtgcagcttgtaagttcttgaaaagaagatgcactcccaattgcatatttgctccatattttcgctcagatgaacctaaaaagtttgccagagtacacaaggtttttggtgcaagcaatgtgagcaagatactgattgaagtgcccgaagagcaacgtgaagacacggtgaattctctagcttatgaggctgaggcaaggcttacagaccctgtctacggttgcattggtgctatcgctctgttgcaaagaaaaatggttgagctacaagttgatcttgccattgctagagctcgtctagctcggtatgctgccaattctcctcctatcttgaatgatcatggtagcatgataccaaccttggctgaattccctgcttgtggtggattggttgacagttttaaccagagttcatccgataccatgaatgatttcagccaattcccatttatatcttaa

SEQ ID NO: 8 - DNA, 735 bp, Populus trichocarpa
atgagtactacaacaacaaaccctataaatagcggtgttggtggtgggagcagtggtggtggaggtgcaggtggaagtagtggtggtggaggtgggccatgtggtgcttgtaaattcttgaggaggaagtgtgtgccaggctgtatatttgcaccttattttgactcggagcaaggtgcggcacattttgcagcggtgcataaggtttttggtgcgagtaatgtgtcaaagcttttattacacatacctgcccacaaaagacctgatgctgtggtcacaatttgctatgaggctcaagctcgtttaagagatccagtttatggatgtgttgctcacatctttgctcttcaacaacaggtggtaaatttacaagcagaactctcatacttacaagcccatctagcagcaatggaagttccatcgccacctcctccaccaccgccagcactagtgacgtcgcctccattctcaatagctgacctcccatcagcctcttctatccccggcgccgcgtatgatttatcatctctttttgatccaatggtgcaaccatcatggtccatgcagccaagacagctggacccccatcagtttggaggcagcagtggcacgtcagaaactagtggtggtgatctacaagcattggctcgtgagctcctccatagacgaggatctctaccgccagggtccgtgccctgtagtgacgcattagcatcatcatctatctctaaatga

SEQ ID NO: 9 - protein, 213 aa, Populus trichocarpa
MDMIEQSAAISPINVPSQFSYSPSSSSPPSSHHSSPSKSSPNLTPPSAATPPPPPPVVSPCAACKILRRRCVDKCVLAPYFPPSEPYKFTIAHRVFGASNIIKFLQELPESQRADAVSSMVYEANARIRDPVYGCAGAISQLQKQVSDLQAQLAKAQAEVVNMQCQQANLVALLCMEMTQSQQEPILQQHQYVDTSCFLDENNLGTSWEPLWT

SEQ ID NO: 10 - protein, 231 aa, Populus trichocarpa
MRMSCNGCRILRKGCGDNCSIKPCLQWIETPDSQANATLFLAKFYGRAGLMNLINACPQHLRPDTFKSLLYEACGRIVNPVSGSVGLLSTGSWQQCQAAVEAVLKGEPITQIASTDQLTLGGSDTRHVSREEDWSASDQPRKIKSKRQFNRSTSKRKPSRTEAEHTCFEFMIGFDGLRSVSPDSVLSWRPSLGSDNEETDSIGSVETVEASKLDEPAEGSDLDLDLTLGHY

SEQ ID NO: 11 - protein, 146 aa, Populus trichocarpa
MKESGRKLGALSPCAACKLLRRRCAQDCMFAPYFPADEPQKFASVHKVFGASNVNKMLQELPEHQRSDAVSSMVYEANARVRDPVYGCVGAISSLQKQIDSLQTQLAIAQAEVVHMRVRQFTSSSNPGVMDMAVDQATMGESLWSC

SEQ ID NO: 12 - protein, 233 aa, Populus trichocarpa
MRMSCNGCRVLRKGCSENCSIRPCLQWIKSSESQANATVFLAKFYGRAGLMNLINAGPEHLRPAIFRSLLYEACGRIVNPIYGSVGLMWSGSWQLCQAAVEAVLKGAPITPINSEAAVNGHGPPLKVYDIRHVSKEENSAASNDANRARTRCRSSVSHQSELAVLDGDSKESDESMAHDVASNGISGLELGLDLTLGLEPVSRAHHVVPVKKRRVEAYGSGDVDTCKMELRLE

SEQ ID NO: 13 - protein, 246 aa, Populus trichocarpa
MSTATTNPINTGAGGGSSGGGGGGGGGGGGGGGPCGACKFLRRKCVPGCIFAPYFDSEQGAAHFAAVHKVFGASNVSKLLLHIPVHKRLDAVVTICYEAQARLRDPVYGCVAHIFALQQQVVNLQAELSYLQAHLAALEVPSPPPPPPPTLVTPPPLSIADLPSASSIPAAYDLSCLFDPMMQPSWSNMQPQRQMDIRQFGGSGGSSGTGGGDLQALARELLHRRGSPPPGFMSCSDTLASPSISK

SEQ ID NO: 14 - protein, 223 aa, Populus trichocarpa
MSRDRERSEELGKRIKRESDASSFMGRRQMLGPPVILNTVTPCAACKLLRRRCAEECPFSPYFSPHEPQKFAAVHKVYGASNVSKLLMEVPESQRVDTANSLVYEANLRLRDPVYGSMGAISALQQQIQSLQAELSAIRAEILNYKYREAAAATNIISSTHPALVSSATVSISTPSQTLAPPPQPPPPSVVVSSSSSSSLYTPPTSTSGYSTISSENNVPYFD

SEQ ID NO: 15 - protein, 163 aa, Populus trichocarpa
MRNQEPRASSSCAACKFLKRRCTPNCIFAPYFRSDEPKKFARVHKVFGASNVSKILIEVPEEQREDTVNSLAYEAEARLTDPVYGCIGAIALLQRKMVELQVDLAIARARLARYAANSPPILNDHGSMIPTLAEFPACGGLVDSFNQSSSDTMNDFSQFPFIS

SEQ ID NO: 16 - protein, 244 aa, Populus trichocarpa
MSTTTTNPINSGVGGGSSGGGGAGGSSGGGGGPCGACKFLRRKCVPGCIFAPYFDSEQGAAHFAAVHKVFGASNVSKLLLHIPAHKRPDAVVTICYEAQARLRDPVYGCVAHIFALQQQVVNLQAELSYLQAHLAAMEVPSPPPPPPPALVTSPPFSIADLPSASSIPGAAYDLSSLFDPMVQPSWSMQPRQLDPHQFGGSSGTSETSGGDLQALARELLHRRGSLPPGSVPCSDALASSSISK

(19) United States
(12) Patent Application Publication
(10) Pub. No.: US 2012/0185968 A1
(43) Pub. Date: Jul. 19, 2012

(54) PLANTS OVEREXPRESSING SEQUENCES THAT PROMOTE WOOD PRODUCTION

(75) Inventors: Victor B. Busov, Houghton, MI (US); Yordan S. Yordanov, Houghton, MI (US)

(73) Assignee: MICHIGAN TECHNOLOGICAL UNIVERSITY, Houghton, MI (US)

(21) Appl. No.: 13/497,426

(22) PCT Filed: Sep. 27, 2010
(86) PCT No.: PCT/US10/50371

§ 371 Date: Mar. 21, 2012

Related U.S. Application Data
(60) Provisional application No. 61/246,000, filed on Sep. 25, 2009.

(51) Int. Cl. A01H 5/00; C12N 15/82; C07K 14/415; C12N 15/29; C12N 5/10

(52) U.S. Cl. 800/290; 536/23.6; 435/320.1; 435/419; 800/298; 530/370; 800/278

(57) ABSTRACT

Polynucleotides encoding polypeptides that increase the secondary growth of plants were identified. Introduction of the polynucleotides into plants produces plants having altered characteristics, such as increased secondary growth, increased phloem production, increased xylem production, increased number of internodes, and/or increased stem diameter. Expression of the polynucleotides in plants in the antisense orientation may produce plants that have decreased secondary growth, decreased phloem production, decreased xylem production, decreased numbers of internodes, and/or decreased stem diameter.

### CROSS-REFERENCE TO RELATED APPLICATIONS

This application claims the benefit of priority to U.S. Provisional Patent Application No. 61/246,000, the entire content of which is hereby incorporated by reference in its entirety for all purposes.

### STATEMENT REGARDING FEDERALLY SPONSORED RESEARCH

This invention was made with United States government support from the Department of Energy grant number E21788. The United States government has certain rights in this invention.

### INTRODUCTION

Modified plants having altered characteristics such as increased wood production would be useful for the paper and biofuels industries by providing plants having a greater proportion of wood relative to other parts of the plant.

### SUMMARY

An isolated polynucleotide comprising a contiguous coding sequence encoding a polypeptide comprising an amino acid sequence having at least 95% identity to at least one sequence selected from SEQ ID NOs: 1-8, and plants and plant cells containing such polynucleotides, are provided. In certain embodiments, a plant comprising the isolated polynucleotide exhibits increased expression of the polypeptide, relative to a control plant, and may exhibit increased secondary growth. Such increased secondary growth may be, for example, increased phloem production and/or increased xylem production. In certain embodiments, such increased secondary growth results in a plant with a greater proportion of wood relative to a control plant. In certain embodiments, a plant comprising the isolated polynucleotide exhibits an increased number of internodes relative to a control plant.

In certain embodiments, an isolated polypeptide comprising an amino acid sequence having at least 95% identity to at least one sequence selected from SEQ ID NOs: 1-8 is provided.

In certain embodiments, methods of producing transgenic plants by introducing into a plant cell a polynucleotide encoding a polypeptide comprising an amino acid sequence having at least 95% identity to at least one sequence selected from SEQ ID NOs: 1-8, and regenerating the transformed cell to produce a transgenic plant, are provided. In certain embodiments, the transgenic plant exhibits increased secondary growth. Such increased secondary growth may be, for example, increased phloem production and/or increased xylem production. In certain embodiments, such increased secondary growth results in a plant with a greater proportion of wood relative to a control plant. In certain embodiments, the transgenic plant exhibits an increased number of internodes relative to a control plant.

In certain embodiments, methods of producing transgenic plants by introducing into a plant cell a polynucleotide comprising a sequence having at least 95% identity to the complement of at least one sequence selected from SEQ ID NOs: 1-8 and regenerating the transformed cell to produce a transgenic plant are provided. In certain embodiments, a transgenic plant containing the polynucleotide exhibits decreased secondary growth relative to a control plant. In certain embodiments, a transgenic plant containing the polynucleotide exhibits decreased phloem and/or xylem production. In certain embodiments, a transgenic plant containing the polynucleotide exhibits a decreased number of internodes relative to a control plant.

### BRIEF DESCRIPTION OF THE FIGURES

FIG. 1. Morphological changes in mutant (A, C, E) vs. WT-717 (B, D, F) plants. A and B show the difference in bark texture; C and D show increased phloem proliferation in mutant plants; E and F show the leaf morphology of mutant and wild-type plants. Arrows point to the number of layers with phloem bundles in mutant (C) and WT (D) plants.

FIG. 2. Position of the tag (A) and expression of the two most proximal genes (B) is shown. Yellow arrows represent the proximal genes and the red arrow represents the enhancer tetramere in the tag. Two loading controls (ubiquitin and cyclophilin) are included in (B).

FIG. 3. Recapitulation of mutant phenotype via retransformation. In all photos (A-D) a typical transgenic (P35S) plant is shown on the left and a typical WT-717 is shown on the right (Photos taken of multiple representative plants in the phenotypic class). E, F, and G show various growth parameters measured in approximately 2-month-old greenhouse-grown plants. Asterisks indicate significance as determined by student t-test with ‘**’ and ‘*’ denoting P<0.01 and P<0.05, respectively.

FIG. 4. Increased phloem proliferation in mutant plants (B, E, H) and recapitulated mutant plants (C, F, I) as compared to WT-717 plants (A, D, G). Representative sections were taken from two-month-old, greenhouse-grown plants. All plants were grown for the same period, under the same conditions and sampled at the same time at the respective internodes indicated on the figure.

FIG. 5. Rays initiation and proliferation. Typical ray cells density in WT-717 plants (A), mutant plants (B), and recapitulated mutant plants (C). Rays density was quantified (D) in multiple sections as shown in A-C. Biseriate (E) and multiseriate (F, G) rays can be frequently observed in mutant plants and P35S::PtaLBD11 (recapitulated mutant) plants. Arrows point to thickened biseriate rays.

FIG. 6. Phylogenetic relationships of the LBD gene family in poplar and Arabidopsis. Arrow points to the activation tagged gene PtaLBD11.

FIG. 7. Expression of 53 putative poplar LBD genes studied by Affymetrix gene chip. A. Expression of 53 putative poplar LBDs represented by 61 array probesets in 7 tissues: apex (A), young leaf (YL), mature leaf (ML), primary stem from the top (TS), stem from the bottom (BS), xylem (X), and root (R). ‘Top stem’ refers to top internodes with no signs of vascular cambium and secondary growth while ‘bottom stem’ refers to stems that undergo secondary woody growth with well-developed vascular cambium. B and C show genes that are preferentially expressed in secondary stems with high (B) and low (C) expression in xylem. We hypothesize that low expression in xylem (C) suggests phloem-specific expression similar to expression of phloem-market gene APL (Bonke, M. et al. Nature (2003) 426(6963):181-6; Zhao, C. et al. Plant Physiol. (2005) 138(2):803-18 (Epub May 27, 2005)) (inset in C). Note contrasting pattern of xylem marker gene (inset in B) encoding cellulose IRX3-CesA7-A (Kumar, M. et al. Trends in Plant Science (2009) 14:248-254; Zhao, C. et al. Plant Physiol. (2005) 138(2):803-18 (Epub May 27, 2005)).

FIG. 8. RT-PCR expression of poplar LBD genes in developing xylem and phloem-bark tissues. Left diagram shows semiquantative expressions of the genes. The bars represent standard error from two biological replications, and gene expression was normalized to ubiquitin. At right are representative pictures of RT-PCR amplification of the genes. APL is a phloem-marker gene (Bonke, M. et al. Nature (2003) 426(6963):181-6; Zhao, C. et al. Plant Physiol. (2005) 138(2):803-18 (Epub May 27, 2005)) and IRX3 is a xylem marker gene encoding cellulase IRX3-CesA7-A (Kumar, M. et al. Trends in Plant Science (2009) 14:248-254; Zhao, C. et al. Plant Physiol. (2005) 138(2):803-18 (Epub May 27, 2005)).

FIG. 9. RT-PCR expression of 4 LBD genes (A) Predominant expression of 4 LBD genes (see Table 1) during secondary growth. PSt—stems from internodes 1-8, SSt—stems from internodes 10-20. (B). Differential expression of the selected genes in secondary phloem and xylem. Data in (A) and (B) represents relative expression (mean±SE) from three biological replications normalized to expression of an ubiquitin loading control gene.

### DETAILED DESCRIPTION

The present disclosure relates to polynucleotides and polypeptides and use of the polynucleotides and polypeptides for modifying the phenotype of plants or plant cells. Modified plants or plant cells comprising the polynucleotides and/or polypeptides are also provided. In certain embodiments, the modified plants or plant cells exhibit increased secondary growth compared with control plants or plant cells. In certain embodiments, the modified plants or plant cells exhibit increased phloem and/or xylem production, and/or an increased number of internodes, relative to control plants. The modified plants may exhibit increased stem diameter, in certain embodiments, relative to control plants. Stem diameter may be measured, for example, at the base of the plant.

The polypeptides discussed herein are termed Lateral Organ Boundary domain (LBD) proteins and show homology to certain LBD protein sequences from Arabidopsis thaliana. The LBD proteins from poplar are termed PtaLBD. The term “LBD,” however, is used generically to refer to LBD proteins from any species, including poplar.

In certain embodiments, increasing the expression of an LBD protein having an amino acid sequence with at least 95% identity to SEQ ID NOs: 9-16 in plants (for example, by introducing a polynucleotide sequence having at least 95% identity to a sequence selected from SEQ ID NOs: 1-8 into the plant) results in plants that exhibit increased secondary growth, including, but not limited to, increased phloem production and/or increased xylem production, relative to control plants in which expression of the LBD polypeptide has not been increased. Increasing the expression of such an LBD protein may also, in certain embodiments, result in plants that have increased numbers of internodes relative to control plants. Increasing the expression of such an LBD protein may also, in certain embodiments, result in plants that have increased stem diameter relative to control plants. Stem diameter may be measured, for example, at the base of the plant.

In certain embodiments, increasing the expression of, or ectopically expressing, at least one LBD protein selected from LBD11, LBD41, and LBD4 results in plants that exhibit increased phloem production, where the terms LBD11, LBD41, and LBD4 are used generically to refer to a protein from any plant species having homology to Arabidopsis thaliana LBD11, LBD41, and LBD4, respectively. In certain such embodiments, increasing the expression of, or ectopically expressing, at least one LBD protein selected from PtaLBD11, PtaLBD41a, PtaLBD41b, and PtaLBD4 results in plants that exhibit increased phloem production. Such plants, in certain embodiments, may also exhibit increased xylem production, e.g., as a compensating effect. Further, in certain embodiments, increasing the expression of, or ectopically expressing, at least one LBD protein selected from LBD30, LBD15, LBD21, and LBD18 results in plants that exhibit increased xylem production, where the terms LBD30, LBD15, LBD21, and LBD18 are used generically to refer to a protein from any plant species having homology to Arabidopsis thaliana LBD30, LBD15, LBD21, and LBD18, respectively. Thus, in certain embodiments, increasing the expression of, or ectopically expressing, at least one LBD protein selected from PtaLBD30, PtaLBD15, PtaLBD21, and PtaLBD18 results in plants that exhibit increased xylem production. Such plants, in certain embodiments, may also exhibit increased phloem production, e.g., as a compensating effect.

As used herein, “ectopically expressing” a polynucleotide or polypeptide refers to any method of expressing a polynucleotide from a location other than its normal genomic location in a cell. Such ectopic expression includes, but is not limited to, expression from a DNA element, such as a transgene or virus, that integrates into the genome of the cell, and expression from extragenomic DNA, including, but not limited to, vectors, plasmids, viruses, etc. When stable expression is desired, in certain embodiments, a DNA element that integrates into the genome is used. The polynucleotide being ectopically expressed may be a polynucleotide encoding a polypeptide that is native to the plant species in which it is being ectopically expressed, or it may encode a polypeptide that is not native, such as, for example, a homologous polypeptide from another plant species. Thus, in certain embodiments, an LBD polynucleotide from one plant species may be transformed into another plant species to carry out the methods described herein.

Certain exemplary LBD polynucleotide sequences include, but are not limited to, the sequences of SEQ ID NOs: 1-8, polynucleotide sequences that encode the amino acid sequences of SEQ ID NOs: 9-16, and polynucleotide sequences that encode amino acid sequences having at least 95% identity to at least one amino acid sequence selected from SEQ ID NOs: 9-16.

Certain exemplary LBD polynucleotides also include, but are not limited to, polynucleotides that encode a polypeptide comprising an amino acid sequence having at least about 80%, at least about 85%, at least about 90%, at least about 95%, at least about 98%, or at least about 99% identity to at least one amino acid sequence selected from SEQ ID NOs: 9-16. Percent identity may be determined using the algorithm of Altschul et al., Nucleic Acids Res. 25:3389-3402 (1997). Such an algorithm is incorporated into the BLASTP program, which may be used to obtain amino acid sequences homologous to a reference polypeptide, as is known in the art. In various embodiments, the polynucleotide is an isolated polynucleotide, a recombinant polynucleotide, and/or a synthetic polynucleotide. In certain embodiments, a polynucleotide comprises a contiguous coding sequence encoding a polypeptide comprising an amino acid sequence having at least about 80%, at least about 85%, at least about 90%, at least about 95%, at least about 98%, or at least about 99% identity to at least one amino acid sequence selected from SEQ ID NOs: 9-16. As used herein, “contiguous” means that the nucleotides of the coding sequence are connected in an unbroken sequence (for example, the coding region of the polynucleotide lacks introns).

Certain exemplary LBD polypeptides include, but are not limited to, polypeptides comprising an amino acid sequence selected from SEQ ID NOs: 9-16, but having one or more conservative amino acid substitutions. LBD polynucleotides encoding such polypeptides are also contemplated.

As used herein, “polynucleotide” includes reference to a deoxyribonucleotide or ribonucleotide polymer in either single- or double-stranded form. The use of the terms “polynucleotide constructs” or “nucleotide constructs” herein is not intended to limit the present invention to nucleotide constructs comprising DNA. Polynucleotide constructs and oligonucleotides composed of ribonucleotides and combinations of ribonucleotides and deoxyribonucleotides, may also be employed in the methods disclosed herein. The nucleotide constructs, nucleic acids, and nucleotide sequences of the invention additionally encompass all complementary forms of such constructs, molecules, and sequences.

Transgenic plants and methods of producing transgenic plants are provided. Such transgenic plants are produced, in certain embodiments, by introducing into a plant or plant cell a polynucleotide encoding a polypeptide comprising a sequence having at least about 80%, at least about 85%, at least about 90%, at least about 95%, at least about 98%, or at least about 99% identity to at least one amino acid sequence selected from SEQ ID NOs: 9-16. In certain embodiments, the polynucleotide is provided as a construct in which a promoter is operably linked to the polynucleotide. The promoter is suitably a heterologous promoter. A heterologous promoter includes, for example, a promoter which is different from that naturally associated with and promoting expression of the polynucleotide, or a modified variant of the polynucleotide's own promoter. Such transgenic plants may also be produced, in certain embodiments, by introducing into a plant or plant cell a polynucleotide encoding a polypeptide comprising a sequence having at least about 80%, at least about 85%, at least about 90%, at least about 95%, at least about 98%, or at least about 99% identity to at least one protein selected from LBD30, LBD15, LBD21, LBD18, LBD11, LBD41, and LBD4.

It is envisaged that a plant produced following the introduction of such a polynucleotide exhibits altered or modified characteristics. The modified characteristics include, but are not limited to, increased secondary growth, such as increased phloem production and/or increased xylem production relative to control plants. The modified characteristics may also include, but are not limited to, increased number of internodes relative to control plants, and/or increased stem diameter relative to control plants. As a nonlimiting example, such modified plants may have a stem diameter that is at least about 20%, at least about 30%, at least about 50%, at least about 75%, or at least about 100% greater than the stem diameter of a control plant.

As used herein, a “control plant” is a plant that is substantially equivalent to a test plant or modified plant in all parameters with the exception of the test parameters. For example, when referring to a plant into which a polynucleotide according to the present invention has been introduced, in certain embodiments, a control plant is an equivalent plant into which either no such polynucleotide has been introduced. In certain embodiments, a control plant is an equivalent plant into which a control polynucleotide has been introduced. In such instances, the control polynucleotide is one that is expected to result in little or no phenotypic effect on the plant.

The polynucleotides of the present invention may be introduced into a plant cell to produce a transgenic plant. As used herein, “introduced into a plant” with respect to polynucleotides encompasses the delivery of a polynucleotide into a plant, plant tissue, or plant cell using any suitable polynucleotide delivery method. Methods suitable for introducing polynucleotides into a plant useful in the practice of the present invention include, but are not limited to, freeze-thaw method, microparticle bombardment, direct DNA uptake, whisker-mediated transformation, electroporation, sonication, microinjection, plant virus-mediated, and Agrobacterium-mediated transfer to the plant. Any suitable Agrobacterium strain, vector, or vector system for transforming the plant may be employed according to the present invention.

In some embodiments, a plant may be regenerated or grown from the plant, plant tissue or plant cell. Any suitable methods for regenerating or growing a plant from a plant cell or plant tissue may be used, such as, without limitation, tissue culture or regeneration from protoplasts. Suitably, plants may be regenerated by growing transformed plant cells on callus induction media, shoot induction media and/or root induction media.

In certain embodiments, the polynucleotides to be introduced into the plant are operably linked to a promoter sequence and may be provided as a construct. As used herein, a polynucleotide is “operably linked” when it is placed into a functional relationship with a second polynucleotide sequence. For instance, a promoter is operably linked to a coding sequence if the promoter is connected to the coding sequence such that it may effect transcription of the coding sequence. In various embodiments, the polynucleotides may be operably linked to at least one, at least two, at least three, at least four, at least five, or at least ten promoters.

Promoters useful in the practice of the present invention include, but are not limited to, constitutive, inducible, temporally-regulated, developmentally regulated, chemically regulated, tissue-preferred and tissue-specific promoters. Suitably, the promoter causes sufficient expression in the plant to produce the phenotypes described herein. Suitable promoters include, without limitation, the 35S promoter of the cauliflower mosaic virus, ubiquitine, tCUP cryptic constitutive promoter, the Rsyn7 promoter, pathogen-inducible promoters, the maize In2-2 promoter, the tobacco PR-1a promoter, glucocorticoid-inducible promoters, and tetracycline-inducible and tetracycline-repressible promoters.

Polynucleotides may also be provided in a vector. Suitable vectors include plasmids and virus-derived vectors. Vectors known in the art that are suitable for transformation into plants, cloning, and protein expression may be used.

Isolated polypeptides are also provided. In certain embodiments, an isolated polypeptide comprises an amino acid sequence having at least about 80%, at least about 85%, at least about 90%, at least about 95%, at least about 98%, or at least about 99% identity with at least one amino acid sequence selected from SEQ ID NOs: 9-16. In certain embodiments, an isolate polypeptide comprises an amino acid sequence selected from SEQ ID NOs: 9-16. Such polypeptides may be synthesized and contacted with plants and/or plant cells. Such polypeptides may, in certain embodiments, promote secondary growth of the contacted plants. Accordingly, methods of promoting secondary growth comprising contacting plants with a polypeptide comprising an amino acid sequence having at least about 80%, at least about 85%, at least about 90%, at least about 95%, at least about 98%, or at least about 99% identity with at least one amino acid sequence selected from SEQ ID NOs: 9-16 are provided.

Methods of increasing secondary growth, including for example, increasing phloem production and/or xylem production, in plants are provided. Such methods comprise, in certain embodiments, contacting one or more plant cells with one or more isolated LBD polypeptides, or otherwise effecting an increase in the amount or concentration of the LBD polypeptide in the plant cell, such as by introducing a polynucleotide encoding the LBD polypeptide into the cell. The polynucleotide may be introduced in a vector or construct and may be expressed transiently. Plant cells may also be transformed with polynucleotide sequences encoding polypeptides of the invention, such that the polynucleotide stably integrates into the genome or chromosomes of a plant cell.

The plants that can be used in the methods described herein include any woody plants. By “woody plants” is meant plants that exhibit secondary growth originating in the cambium. Suitable woody plants include, but are not limited to, shrubs, vines, or trees such as aspen, fir, maple, acacia, box elder, horse chestnut, buckthorn, buckeye, mimosa, alder, birch, hornbeam, hickory, chestnut, cedar, red bud, cypress, buck wheat, dogwood, hawthorn, persimmon, olive, eucalyptus, rubber, euonymus, beech, ash, witch-hazel, holly, juniper, myrtle, larch, sweet gum, poplar, oak, magnolia crabapple, redwood, spruce (Norway spruce, dragon spruce, white spruce, black spruce, Colorado blue spruce, red spruce, Himalayan spruce), pine (bristle cone pine, weston white pine, longleaf pine, ponderosa pine, scotch pine, loblolly pine), sycamore, plane, cottonwood, poplar, plum, cherry, laurel, peach, Douglas fir, sumac, willow, elderberry, mountain ash, bladdernut, yew, linden, hemlock, and elm.

As used herein, the term “plant” includes reference to whole plants, plant organs (e.g., leaves, stems, roots, etc.), seeds, plant cells, and progeny of same. Parts of transgenic plants comprise, for example, plant cells, protoplasts, tissues, callus, embryos as well as flowers, ovules, stems, fruits, leaves, roots originating in transgenic plants or their progeny previously transformed with a DNA. As used herein, the term “plant cell” includes, without limitation, protoplasts and cells of seeds, suspension cultures, embryos, meristematic regions, callus tissue, leaves, roots, shoots, gametophytes, sporophytes, pollen, and microspores.

Methods of producing a transgenic plant by introducing in to a plant or plant cell a polynucleotide comprising a sequence having at least about 80%, at least about 85%, at least about 90%, at least about 95%, at least about 98%, or at least about 99% identity to the complement of at least one of SEQ ID NOs: 1-8 are provided. A transgenic plant may be regenerated from the transformed plant or plant cell. In certain embodiments, the polynucleotide is operably linked to a promoter functional in the plant. In certain embodiments, the transgenic plant exhibits decreased secondary growth relative to a control plant. Such decreased secondary growth may include, but is not limited to, decrease phloem production and/or decreased xylem production. The transgenic plant may also exhibit a decreased number of internodes relative to a control plant.

It will be apparent to those of skill in the art that variations may be applied to the compositions and methods described herein and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

It is to be understood that the invention is not limited in its application to the details of construction and the arrangement of components set forth in the following description. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The following non-limiting examples are purely illustrative.

### EXAMPLES

### Example 1

### Identification of the PtaLBD11 Sequence

Hybrid poplar clone WT-717 (P. alba×P. tremula) was transformed with activation tagging vector pSKI074 using Agrobacterium-mediated transformation. Briefly, Agrobacterium cells carrying the binary vector (such as pV-LEGT02) were grown in luria broth, collected by centrifugation, resuspended in induction medium (MS salts, vitamins, 10 μM AS, 10 mM galactose, 1.28 mM 2-(N-morpholino)ethanesulfonic acid [MES], pH 5.0), and induced at room temperature. Explants were soaked for 10-20 minutes in the bacterial suspension under 0.6-bar vacuum and shaken (50 rpm) at room temperature. The inoculated explants were co-cultivated in dark for 2-3 days at 19-25° C. in callus induction medium (CIM) (MS salts, 0.5 μM benzyladenine, 0.5 μM zeatin, 5 μM naphthalene acetic acid, 5 μM 2,4-Dichlorophenoxyacetic acid, 0.3% gelling agent [such as Phytagar™ from Gibco BRL], 0.1% gelling agent [such as Phytagel™ from Sigma], 1.28 mM 2-(N-morpholino)ethanesulfonic acid, pH 5.8). Explants were cultured for 10-30 days in the dark on CIM with 500 mg/L cefotaxime and 50 mg/L kanamycin. Shoot regeneration was induced on shoot induction medium (SIM) (MS salts, 10 μM benzyladenine, 10 μM zeatin, 1 μM N-acetylaspartate, 0.3% Phytagar (Gibco BRL), 0.1% Phytagel (Sigma), 1.28 mM 2-(N-morpholino)ethanesulfonic acid, pH 5.8) for several weeks to months, and explants were subcultured every 2-4 weeks. Regenerated shoots were further screened for kanamycin resistance by rooting in medium supplemented with 0.5 μM indole-3-butyric acid and 25 mg/L kanamycin.

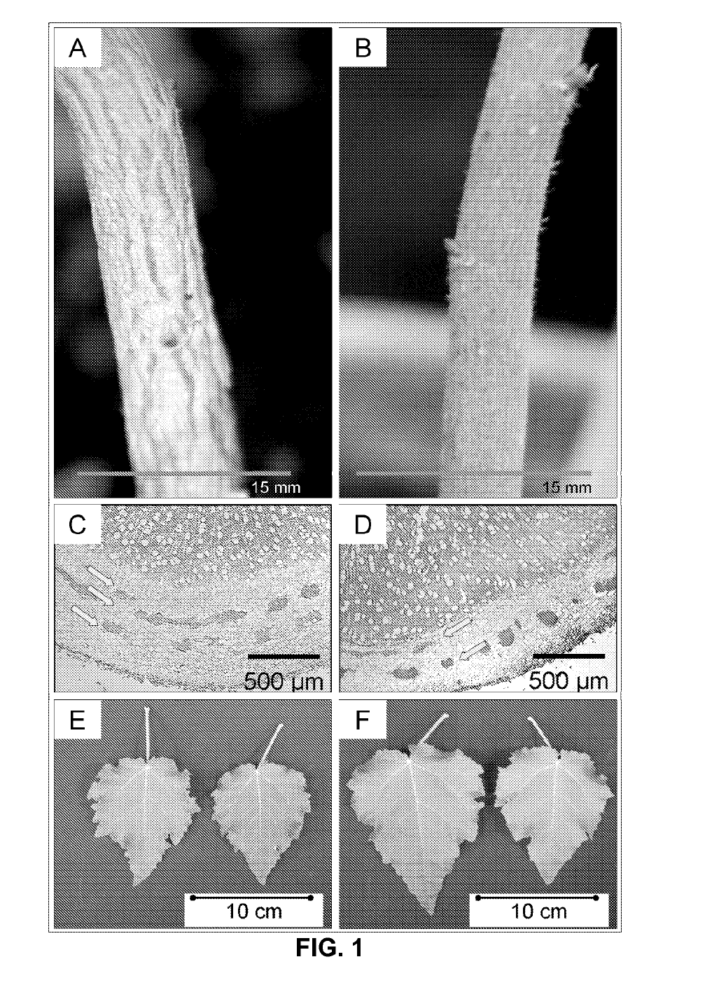

Transgenic lines were recovered after the transformation, and the presence of the activation tagging vector was verified by PCR-amplification using primers specific for the activation tagging vector. We discovered a poplar activation tagged mutant that displayed increased secondary growth and changes in bark texture (FIG. 1). Approximately two-month-old plants had increased stem diameter, deeply-furrowed bark (FIG. 1A) and slightly smaller leaves (FIG. 1E). Although we observed a proportional increase in the production of xylem and phloem, the majority of the girth increase was contributed by increase in phloem production (FIG. 1C). The WT-717 plants at the 25^{th} internode from the top of the plant typically produce two layers of phloem fibers while the mutant displayed three and visible initiation of a fourth was already differentiating along the boundary with the cambium zone (FIG. 1C).

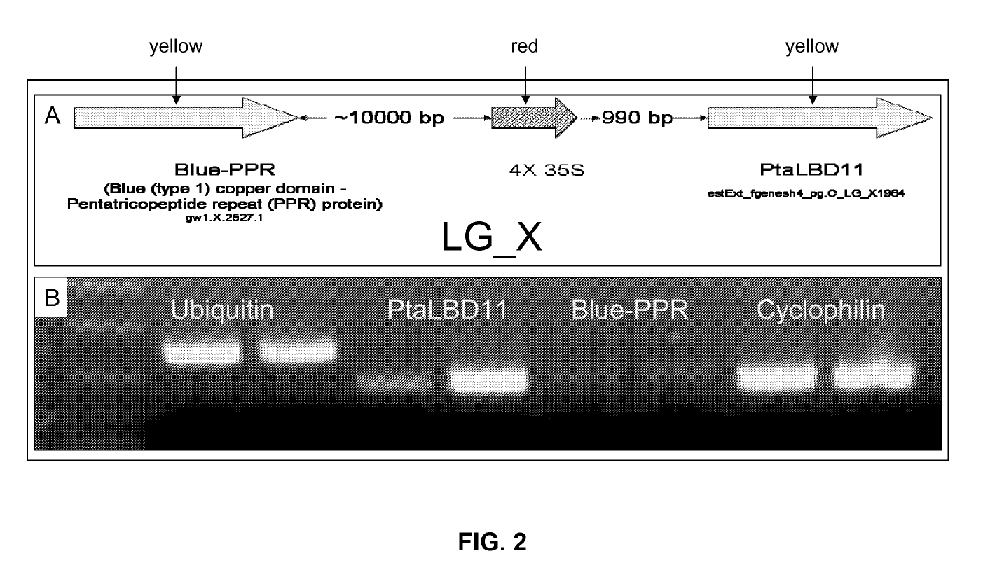

To identify the gene responsible for the observed phenotypic changes, we used modified SiteFinding PCR (Tan, G. H. et al. Nucleic Acids Res. 2005; 33(13):e122) to amplify poplar genomic sequence flanking the T-DNA insertion. We recovered and sequenced ˜150 bp fragment flanking the left border of the vector. Homology searches in the poplar genome positioned the sequence at LG_X:18767889-18768035. Inspection of the genomic region identified two proximal genes (FIG. 2). We used RT-PCR to study the expression of the two genes in the mutant and WT. A clear activation of the gene corresponding to model estExt_fgenesh4_{— —}pg.C_LG_X1964 was observed while the other gene as well as the loading control genes showed no change. Therefore, we hypothesized that the activation of estExt_fgenesh4_pg.C_LG_X1964 is likely the gene responsible for the observed phenotype.

We cloned and sequenced cDNA of the activated gene including the whole open reading frame of 660 bp (219 aa). The putative protein sequence shows high sequence homology to a family of plant-specific transcription factors known as Lateral Organ Boundary domain (LBD) proteins with highest similarity to uncharacterized gene from Arabidopsis annotated as LBD11. Therefore, we named the gene PtaLBD11.

### Example 2

### Introduction of a Vector Comprising the PtaLBD11 Sequence into a Poplar Hybrid

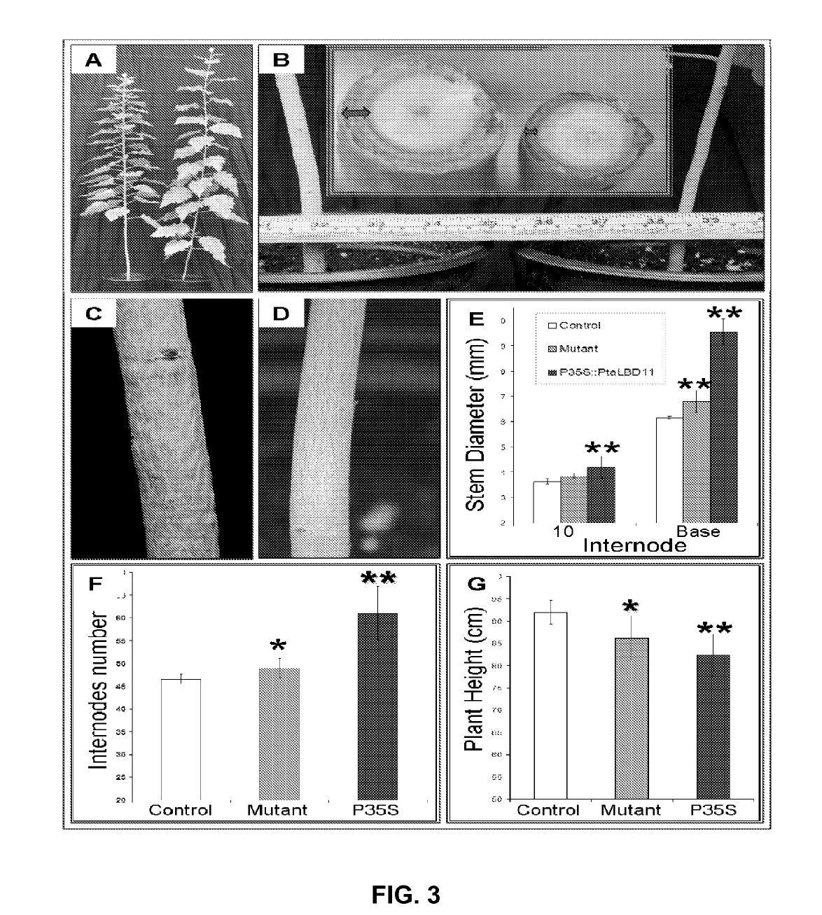

To recapitulate the phenotype, we fused PtaLBD11 to a strong CaMV35S promoter and transformed the construct into the original WT 717-1B4 background where the mutant was discovered. Approximately 20 independent events were recovered, PCR-verified for the presence of the transgene, and overexpression validated by RT-PCR. We observed phenotypic changes very early during the regeneration processes. Most importantly, many of the regenerated shoots appeared to have thick stems. We grew transgenic plants in the greenhouse for approximately two months (FIG. 3A). We observed dramatic changes in bark texture (FIG. 3C) and significant increase in diameter growth (FIGS. 3B, 3E) particularly at the bottom of the wood producing part of the stem (FIG. 3E). These changes were similar to the original mutant but in many cases to a much stronger extent, likely due to high expression from the strong constitutive promoter. As in the original mutant, the leaves were slightly smaller than WT and transgenic plants were shorter. However, because of a significant increase in internode number in transgenic plants, the total height was only slightly affected. Overall, excluding the above-mentioned changes, the transgenic plants were displaying no severe phenotypic abnormalities suggesting a low level of pleitropic effects and highly specific function in wood formation.

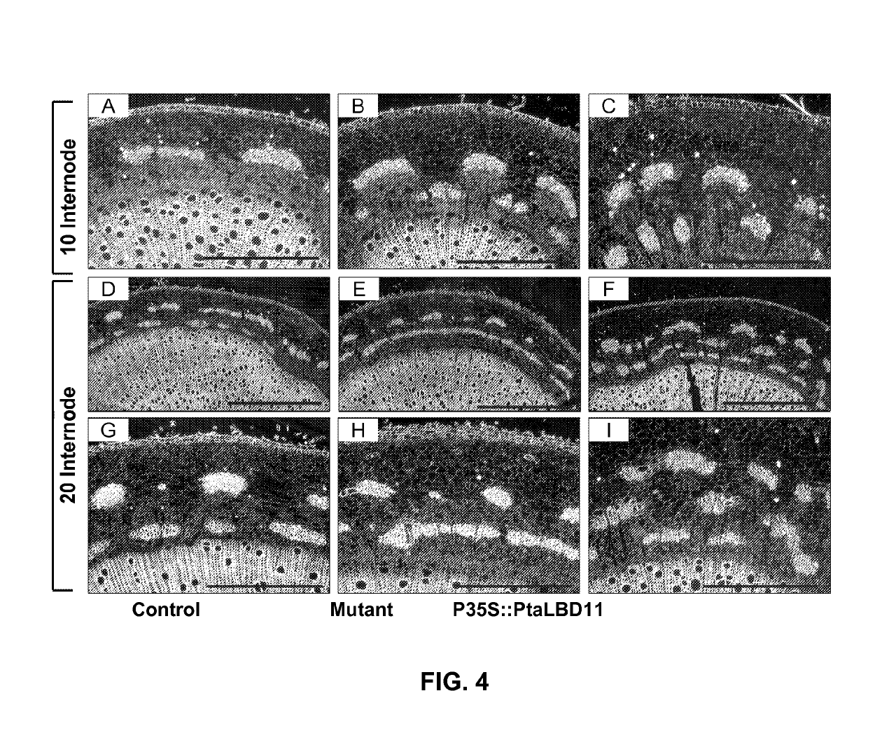

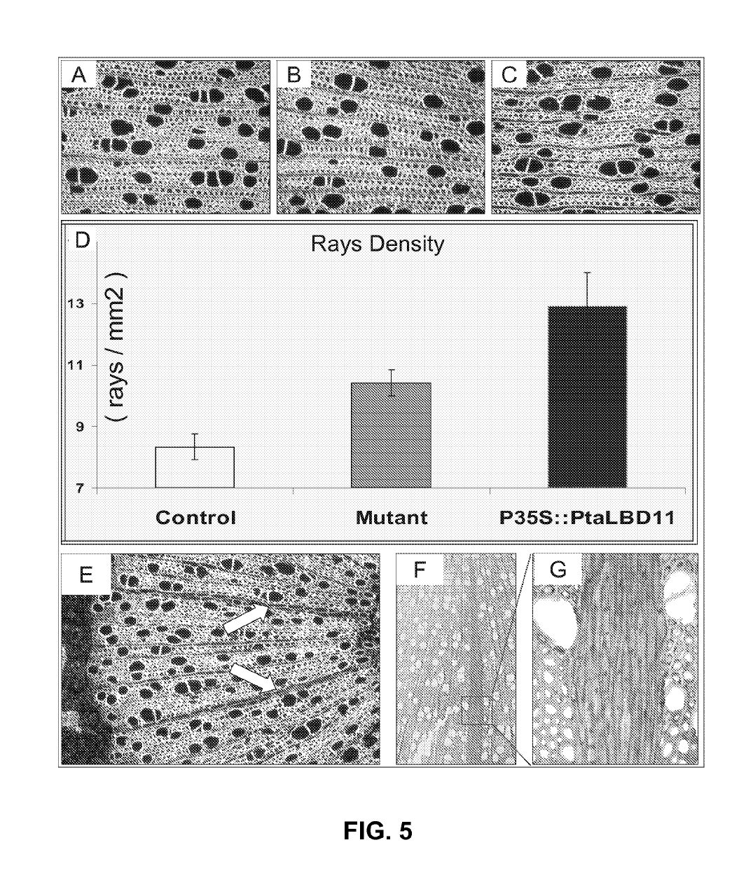

Increased girth growth and changes in the bark texture suggested that the mutation affect wood development. Therefore, we sectioned stems that undergo secondary woody growth. The observed changes were highly consistent with the abnormalities in wood development found in the original mutant. For example, proliferation of secondary phloem was significantly increased and in many cases the enhancement was to a larger extent than in the original mutant (e.g., likely due to strong 35S promoter) (FIG. 4). We also found changes in ray cell formation (FIG. 5). The density of ray cell files was higher in the mutant and 35S::PtaLBD11 plants (FIGS. 5B and C) compared to WT-717 (FIG. 5A). Furthermore, in poplar, rays typically consist of a single cell file (FIG. 5H) and we have never observed deviations from this pattern. In 35S::PtaLBD11 transgenics, we frequently observed instances of biseriate (two rows) (FIG. 5E) and multiseriate (many rows) rays (FIGS. 5F and G) suggesting involvement of this gene in ray cell development.

### Example 3

### Microarray Studies Show LBD Genes Preferentially Expressed During Xylogenesis

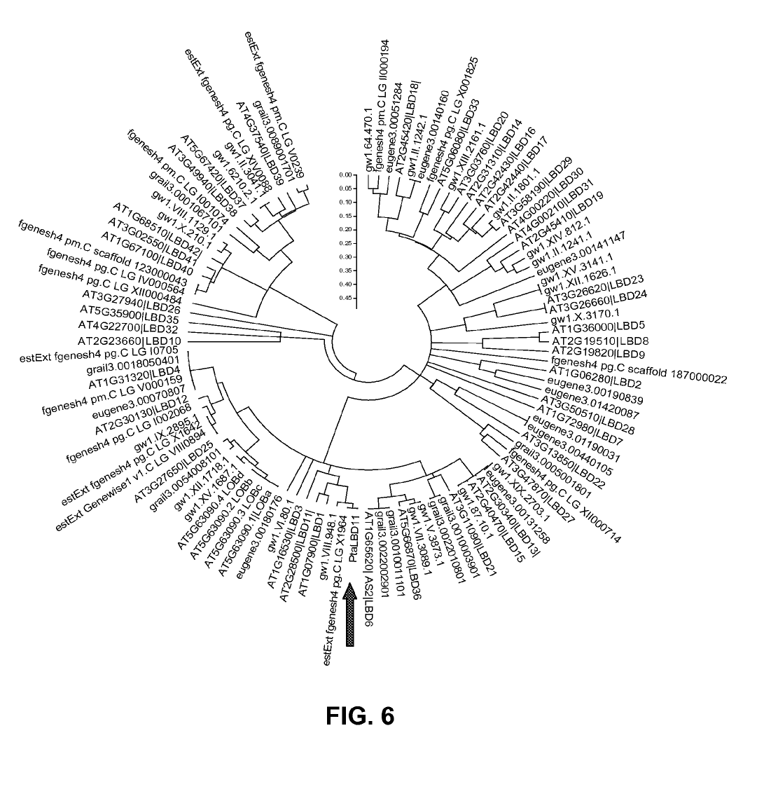

Using homology searches in the poplar genome sequence, we identified 57 genes that encode proteins with similarity to the 42 LBD proteins from Arabidopsis (FIG. 6). As in the Arabidopsis, the Populus genes are grouped in two major classes further subdivided into nine groups (Matsumura, Y et al. Plant J. (2009) 58(3):525-37 (Epub Jan. 19, 2009); Shuai, B. et al. Plant Physiol. (2002) 129(2):747-61). The 1.3× increase in the poplar LBD family is in agreement with a previous report (Zhu, Qi Hui et al. Bioinformatics (2007) 23(10):1307-8 (Epub Mar. 28, 2007)) and almost perfectly matches the overall increase in gene content across the whole poplar genome compared to Arabidopsis and resulting from whole genome duplication in the Salicaceae lineage (Tuskan, G. A. et al. Science (2006) 313(5793):1596-604).

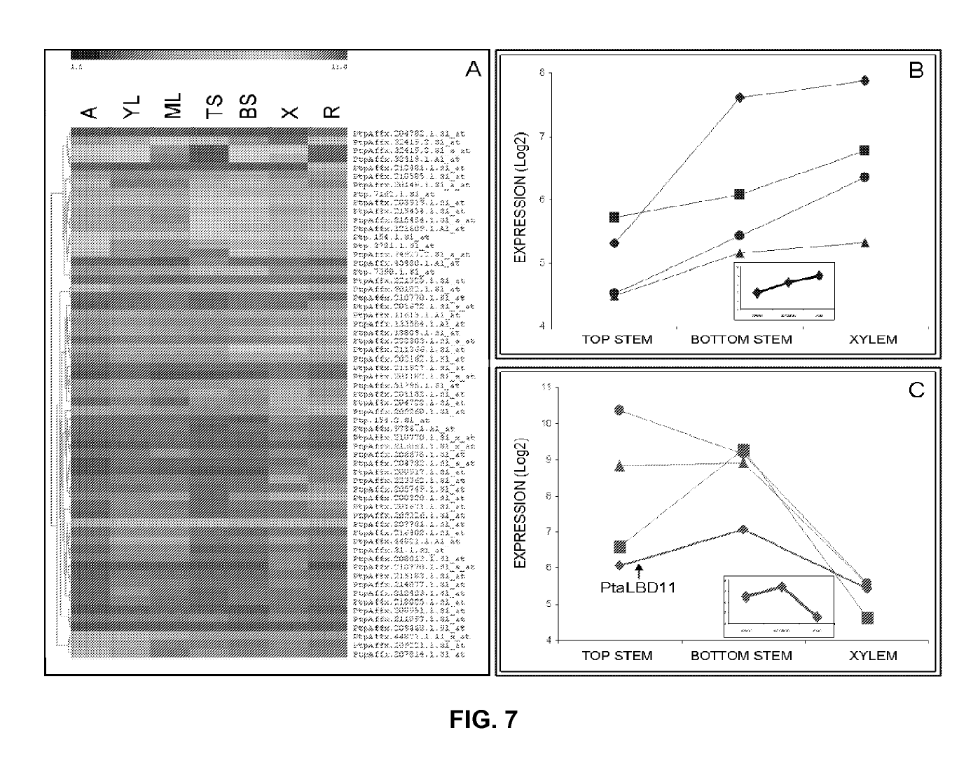

The GeneChip® Poplar Genome Array contains probe sets that correspond to 53 of the 57 LBD genes. We combined our data with publicly available microarray data (Wilkins, O. et al. Plant Physiol. (2009) 149(2):981-93 (Epub Dec. 17, 2008)) to monitor the expression of the 53 LBD genes in seven different plant organs and tissue types (FIG. 7A). The results show highly specific and divergent expression patterns (FIG. 7A).

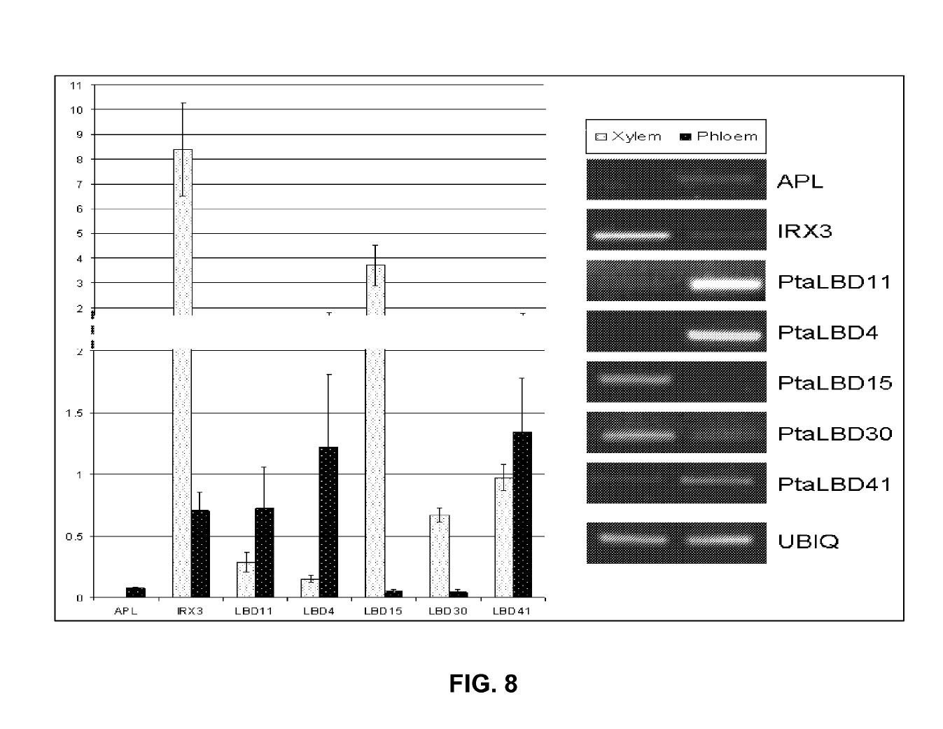

We found eight genes (including the activation tagged PtaLBD 11) that were differentially upregulated (ANOVA, P<0.01) in stems and secondary (woody) xylem (FIGS. 7B and C; see Table 1). Our microarray includes stem samples from the top and bottom of the plant. The stems from the top undergo primary while the ones from the bottom undergo secondary (woody) growth. The xylem tissue was sampled from the bottom of the plant and represents secondary xylem (wood). Among the eight stem-specific LBD genes, there were two easily discernable groups—four expressed (FIG. 7B) and four not expressed (FIG. 8C) in the xylem. Because all of these genes were expressed in wood-forming stems, we believe that absence from the xylem indicates expression in the phloem. In support of this hypothesis, the activation-tagged PtaLBD11 gene showed high expression in the bottom (woody) stem but was nearly absent from the xylem (FIG. 7C), indicating putative phloem-specific expression. This expression pattern was highly consistent with the results obtained from our transgenic work. Both mutant and recapitulation transgenics clearly showed wood changes associated with phloem development (see FIGS. 1 and 4). We also found genes that showed a much higher expression in the bottom (wood-forming) stems and also high expression in xylem suggesting that specific LBD members may play a predominant role in wood formation (FIG. 7B).

In addition, we performed semiquantitive RT-PCR analysis of expression of several of the proposed LBD genes in xylem and phloem-bark tissues (FIG. 8). The result verified the data obtained from the microarray study.

TABLE 1
Proposed function of the LBD genes based on expression analyses

| | Arabidopsis | Function to |
|---|---|---|
| Populus trichocarpa gene model | ortholog | produce: |
| estExt_fgenesh4_pg.C_LG_X1964 | LBD11 | Phloem |
| fgenesh4_pg.C_LG_XII000484 | LBD41a | Phloem |
| eugene3.00070807 | LBD4 | Phloem |
| fgenesh4_pm.C_scaffold_123000043 | LBD41b | Phloem |
| eugene3.00140160 | LBD30 | Xylem |
| eugene3.00131258 | LBD15 | Xylem |
| fgenesh4_pg.C_LG_VIII000617 | LBD21 | Xylem |
| fgenesh4_pm.C_LG_II000686 | LBD18 | Xylem |

TABLE 2
Table of sequences

| SEQ | | POPULUS | |
|---|---|---|---|
| ID | | TRICHOCARPA | |
| NO | DESCRIPTION | GENE MODEL | SEQUENCE |
| 1 | PtaLBD11 | estExt_fgenesh4_p | ATGGATATGATTGAGCAATCTGCAGCAATATCTCCGATC |
| | polynucleotide | g.C_LG_X1964 | AATGTTCCATCTCAATTCTCCTACTCTCCATCCTCTTCT |
| | sequence | | TCACCACCTTCTTCTCATCATTCTTCTCCTTCAAAGTCT |
| | | | TCTCCAAATCTTACTCCACCTTCAGCTGCCACTCCCCCT |
| | | | CCTCCTCCGCCAGTTGTTAGCCCTTGTGCTGCCTGCAAA |
| | | | ATCCTTCGCCGGCGTTGCGTTGACAAGTGTGTTTTAGCT |
| | | | CCTTATTTTCCTCCTTCTGAGCCATACAAGTTCACCATT |
| | | | GCTCATAGGGTGTTTGGAGCCAGCAACATCATCAAGTTT |
| | | | TTGCAGGAACTGCCAGAGTCCCAGAGAGCAGATGCAGTG |
| | | | AGCAGCATGGTTTATGAAGCCAATGCGAGGATCAGAGAC |
| | | | CCGGTTTATGGCTGTGCTGGTGCAATTTCTCAGCTTCAA |
| | | | AAACAAGTTAGTGACCTCCAAGCACAGCTAGCCAAGGCA |
| | | | CAAGCTGAGGTGGTGAACATGCAATGCCAGCAAGCCAAT |
| | | | TTAGTTGCTTTACTCTGCATGGAAATGACACAGTCTCAA |
| | | | CAAGAACCCATCTTGCAACAACACCAATACGTTGACACA |
| | | | AGCTGCTTTCTTGACGAGAACAACTTGGGCACATCATGG |
| | | | GAACCTCTTTGGACATGA |
| 2 | PtaLBD41a | fgenesh4_pg.C_LG | ATGCGGATGAGTTGTAATGGTTGCAGAATCCTTCGCAAG |
| | polynucleotide | _XII000484 | GGCTGTGGTGATAATTGTAGTATAAAACCTTGCCTTCAA |
| | sequence | | TGGATTGAAACTCCCGATTCCCAAGCCAATGCCACCCTC |
| | | | TTCCTTGCTAAATTCTACGGTCGTGCTGGACTCATGAAC |
| | | | CTCATCAATGCTTGCCCTCAGCATCTCCGTCCAGATACG |
| | | | TTTAAGTCCTTATTATACGAGGCATGTGGGCGGATTGTG |
| | | | AATCCAGTTTCGGGATCAGTCGGGTTGTTAAGCACCGGG |
| | | | TCATGGCAGCAATGTCAAGCCGCCGTGGAAGCCGTTCTG |
| | | | AAAGGCGAGCCCATCACTCAAATTGCATCAACTGATCAG |
| | | | CTTACACTAGGCGGCTCTGATACGCGCCACGTGTCAAGA |
| | | | GAGGAGGACTGGTCTGCATCTGATCAGCCCCGTAAAATC |
| | | | AAGTCCAAGCGTCAATTCAATCGATCAACTTCAAAGCGG |
| | | | AAGCCGAGCAGGACTGAGGCTGAGCACACATGTTTCGAG |
| | | | TTCATGATTGGATTTGATGGCTTGCGGTCAGTGAGTCCT |
| | | | GACTCTGTGTTGAGTTGGCGTCCGAGTTTGGGGTCTGAC |
| | | | AATGAGGAAACTGACAGTATAGGGTCTGTGGAGACTGTA |
| | | | GAGGCTTCTAAGCTTGATGAGCCAGCCGAAGGAAGTGAT |
| | | | TTGGATTTAGACTTGACCTTGGGTCATTATTGA |
| 3 | PtaLBD4 | eugene3.00070807 | ATGAAGGAGAGTGGTCGAAAACTTGGTGCACTTTCGCCA |
| | polynucleotide | | TGCGCAGCATGCAAGCTTCTTAGGAGGAGATGTGCTCAA |
| | sequence | | GACTGTATGTTTGCTCCTTACTTTCCAGCTGACGAGCCT |
| | | | CAGAAGTTTGCCAGTGTGCACAAGGTCTTTGGTGCTAGC |
| | | | AATGTCAACAAAATGTTACAGGAATTACCTGAGCACCAA |
| | | | CGAAGTGATGCAGTAAGTTCCATGGTGTATGAAGCAAAT |
| | | | GCAAGGGTTCGTGACCCAGTTTATGGTTGTGTTGGTGCA |
| | | | ATATCATCTTTGCAGAAACAAATTGATTCACTACAAACC |
| | | | CAGTTGGCAATTGCACAAGCTGAGGTAGTGCACATGAGA |
| | | | GTGCGGCAATTCACTTCTTCTTCCAATCCAGGAGTCATG |
| | | | GACATGGCAGTTGATCAGGCCACCATGGGAGAGTCTCTG |
| | | | TGGTCATGCTAG |
| 4 | PtaLBD41b | fgenesh4_pm.C_sc | ATGCGGATGAGTTGTAATGGATGTAGAGTTTTACGCAAA |
| | polynucleotide | affold_123000043 | GGCTGTAGTGAAAACTGTAGTATTAGGCCTTGTTTACAG |
| | sequence | | TGGATCAAGAGTTCTGAGTCTCAAGCCAACGCTACTGTT |
| | | | TTTCTTGCCAAGTTTTATGGCCGTGCTGGCCTTATGAAC |
| | | | CTCATCAACGCTGGCCCTGAACATCTCCGTCCTGCGATT |
| | | | TTTAGGTCGTTGCTCTACGAAGCATGCGGGAGGATAGTG |
| | | | AACCCGATTTATGGCTCGGTTGGGCTGATGTGGTCGGGA |
| | | | AGCTGGCAGCTTTGTCAAGCCGCTGTTGAAGCCGTACTA |
| | | | AAAGGTGCTCCGATAACTCCAATTAACTCTGAAGCTGCA |
| | | | GTTAATGGCCATGGCCCCCCGCTAAAAGTTTATGATATA |
| | | | CGACACGTTTCGAAAGAAGAAAACTCGGCTGCGTCAAAT |
| | | | GATGCAAACCGAGCCAGGACTCGGTGCCGGTCTTCGGTG |
| | | | AGTCACCAATCCGAGTTGGCAGTGTTGGATGGCGACAGC |
| | | | AAAGAGTCTGATGAGAGCATGGCCCATGACGTTGCGAGT |
| | | | AACGGAATTTCTGGGCTTGAACTTGGCCTGGACTTGACC |
| | | | CTGGGCCTTGAGCCCGTGTCACGTGCACATCACGTGGTT |
| | | | CCCGTGAAGAAGAGAAGGGTGGAAGCTTACGGGTCTGGT |
| | | | GATGTTGACACGTGTAAGATGGAGCTCAGACTTGAATAA |
| 5 | PtaLBD30 | eugene3.00140160 | ATGAGTACCGCAACTACAAACCCTATAAATACTGGTGCT |
| | polynucleotide | | GGTGGTGGAAGTAGTGGCGGTGGAGGTGGTGGTGGCGGC |
| | sequence | | GGTGGTGGCGGTGGTGGTGGGCCATGCGGTGCTTGTAAA |
| | | | TTCTTGAGGAGGAAGTGTGTGCCAGGGTGTATATTTGCA |
| | | | CCTTATTTTGACTCAGAGCAAGGTGCAGCACATTTTGCA |
| | | | GCGGTGCATAAGGTTTTTGGTGCAAGTAACGTGTCAAAG |
| | | | CTTTTATTACACATTCCTGTCCATAAAAGACTTGATGCT |
| | | | GTGGTCACAATTTGTTATGAGGCTCAAGCTCGCTTAAGA |
| | | | GATCCAGTCTATGGCTGTGTTGCTCACATCTTTGCTCTT |
| | | | CAACAACAGGTAGTAAATTTACAAGCAGAACTCTCATAC |
| | | | TTGCAAGCCCATCTAGCAGCATTGGAGGTTCCGTCGCCA |
| | | | CCTCCTCCGCCCCCGCCAACACTAGTGACCCCGCCTCCA |
| | | | CTCTCAATAGCAGACCTACCATCAGCCTCTTCAATTCCC |
| | | | GCCGCGTACGATTTATCATGTCTTTTTGATCCAATGATG |
| | | | CAACCCTCATGGTCCAACATGCAGCCGCAGCGGCAAATG |
| | | | GATATCCGTCAGTTTGGAGGCAGCGGTGGCTCATCAGGA |
| | | | ACTGGTGGCGGTGATCTCCAAGCATTGGCGCGTGAACTC |
| | | | CTTCATAGACGAGGATCTCCACCACCCGGGTTCATGTCA |
| | | | TGTAGTGACACATTAGCATCACCATCTATCTCTAAATGA |
| 6 | PtaLBD15 | eugene3.00131258 | ATGTCCAGAGACAGGGAGAGATCTGAAGAGTTAGGAAAG |
| | polynucleotide | | AGGATCAAGAGGGAGAGTGATGCATCTTCTTTTATGGGG |
| | sequence | | AGGAGACAAATGTTAGGTCCTCCTGTAATTTTGAACACT |
| | | | GTTACACCTTGTGCTGCATGTAAACTTTTAAGAAGAAGA |
| | | | TGTGCTGAAGAGTGCCCCTTTTCTCCATATTTTTCTCCA |
| | | | CATGAACCCCAGAAATTTGCTGCTGTCCACAAAGTCTAT |
| | | | GGTGCAAGCAATGTCTCCAAGCTGCTAATGGAGGTGCCA |
| | | | GAAAGTCAAAGAGTTGATACTGCAAATAGTCTAGTTTAT |
| | | | GAAGCAAACTTGAGGCTAAGAGATCCAGTATATGGCTCC |
| | | | ATGGGTGCAATTTCAGCTTTACAACAACAAATTCAATCA |
| | | | TTACAAGCTGAACTTAGTGCAATAAGGGCTGAGATACTC |
| | | | AACTACAAATACAGAGAAGCTGCTGCTGCTACTAACATC |
| | | | ATTTCTTCTACTCATCCTGCTTTAGTTTCTTCTGCGACC |
| | | | GTGTCCATTTCAACACCATCACAAACGCTTGCTCCACCA |
| | | | CCACAACCACCTCCTCCTTCAGTTGTTGTTTCTTCATCT |
| | | | TCCTCTTCTTCTCTTTATACACCACCAACTAGCACATCA |
| | | | GGTTATAGCACTATTTCAAGTGAAAATAATGTCCCATAT |
| | | | TTTGATTAA |
| 7 | PtaLBD21 | fgenesh4_pg.C_LG | ATGAGAAATCAGGAGCCTCGTGCAAGCTCTTCATGTGCA |
| | polynucleotide | _VIII000617 | GCTTGTAAGTTCTTGAAAAGAAGATGCACTCCCAATTGC |
| | sequence | | ATATTTGCTCCATATTTTCGCTCAGATGAACCTAAAAAG |
| | | | TTTGCCAGAGTACACAAGGTTTTTGGTGCAAGCAATGTG |
| | | | AGCAAGATACTGATTGAAGTGCCCGAAGAGCAACGTGAA |
| | | | GACACGGTGAATTCTCTAGCTTATGAGGCTGAGGCAAGG |
| | | | CTTACAGACCCTGTCTACGGTTGCATTGGTGCTATCGCT |
| | | | CTGTTGCAAAGAAAAATGGTTGAGCTACAAGTTGATCTT |
| | | | GCCATTGCTAGAGCTCGTCTAGCTCGGTATGCTGCCAAT |
| | | | TCTCCTCCTATCTTGAATGATCATGGTAGCATGATACCA |
| | | | ACCTTGGCTGAATTCCCTGCTTGTGGTGGATTGGTTGAC |
| | | | AGTTTTAACCAGAGTTCATCCGATACCATGAATGATTTC |
| | | | AGCCAATTCCCATTTATATCTTAA |
| 8 | PtaLBD18 | fgenesh4_pm.C_L | ATGAGTACTACAACAACAAACCCTATAAATAGCGGTGTT |
| | polynucleotide | G_II000686 | GGTGGTGGGAGCAGTGGTGGTGGAGGTGCAGGTGGAAGT |
| | sequence | | AGTGGTGGTGGAGGTGGGCCATGTGGTGCTTGTAAATTC |
| | | | TTGAGGAGGAAGTGTGTGCCAGGCTGTATATTTGCACCT |
| | | | TATTTTGACTCGGAGCAAGGTGCGGCACATTTTGCAGCG |
| | | | GTGCATAAGGTTTTTGGTGCGAGTAATGTGTCAAAGCTT |
| | | | TTATTACACATACCTGCCCACAAAAGACCTGATGCTGTG |
| | | | GTCACAATTTGCTATGAGGCTCAAGCTCGTTTAAGAGAT |
| | | | CCAGTTTATGGATGTGTTGCTCACATCTTTGCTCTTCAA |
| | | | CAACAGGTGGTAAATTTACAAGCAGAACTCTCATACTTA |
| | | | CAAGCCCATCTAGCAGCAATGGAAGTTCCATCGCCACCT |
| | | | CCTCCACCACCGCCAGCACTAGTGACGTCGCCTCCATTC |
| | | | TCAATAGCTGACCTCCCATCAGCCTCTTCTATCCCCGGC |
| | | | GCCGCGTATGATTTATCATCTCTTTTTGATCCAATGGTG |
| | | | CAACCATCATGGTCCATGCAGCCAAGACAGCTGGACCCC |
| | | | CATCAGTTTGGAGGCAGCAGTGGCACGTCAGAAACTAGT |
| | | | GGTGGTGATCTACAAGCATTGGCTCGTGAGCTCCTCCAT |
| | | | AGACGAGGATCTCTACCGCCAGGGTCCGTGCCCTGTAGT |
| | | | GACGCATTAGCATCATCATCTATCTCTAAATGA |
| 9 | PtaLBD11 | estExt_fgenesh4_p | MDMIEQSAAISPINVPSQFSYSPSSSSPPSSHHSSPSKS |
| | polypeptide | g.C_LG_X1964 | SPNLTPPSAATPPPPPPVVSPCAACKILRRRCVDKCVLA |
| | sequence | | PYFPPSEPYKFTIAHRVFGASNIIKFLQELPESQRADAV |
| | | | SSMVYEANARIRDPVYGCAGAISQLQKQVSDLQAQLAKA |
| | | | QAEVVNMQCQQANLVALLCMEMTQSQQEPILQQHQYVDT |
| | | | SCFLDENNLGTSWEPLWT* |
| 10 | PtaLBD41a | fgenesh4_pg.C_LG | MRMSCNGCRILRKGCGDNCSIKPCLQWIETPDSQANATL |
| | polypeptide | _XII000484 | FLAKFYGRAGLMNLINACPQHLRPDTFKSLLYEACGRIV |
| | sequence | | NPVSGSVGLLSTGSWQQCQAAVEAVLKGEPITQIASTDQ |
| | | | LTLGGSDTRHVSREEDWSASDQPRKIKSKRQFNRSTSKR |
| | | | KPSRTEAEHTCFEFMIGFDGLRSVSPDSVLSWRPSLGSD |
| | | | NEETDSIGSVETVEASKLDEPAEGSDLDLDLTLGHY* |
| 11 | PtaLBD4 | eugene3.00070807 | MKESGRKLGALSPCAACKLLRRRCAQDCMFAPYFPADEP |
| | polypeptide | | QKFASVHKVFGASNVNKMLQELPEHQRSDAVSSMVYEAN |
| | sequence | | ARVRDPVYGCVGAISSLQKQIDSLQTQLAIAQAEVVHMR |
| | | | VRQFTSSSNPGVMDMAVDQATMGESLWSC* |
| 12 | PtaLBD41b | fgenesh4_pm.C_sc | MRMSCNGCRVLRKGCSENCSIRPCLQWIKSSESQANATV |
| | polypeptide | affold_123000043 | FLAKFYGRAGLMNLINAGPEHLRPAIFRSLLYEACGRIV |
| | sequence | | NPIYGSVGLMWSGSWQLCQAAVEAVLKGAPITPINSEAA |
| | | | VNGHGPPLKVYDIRHVSKEENSAASNDANRARTRCRSSV |
| | | | SHQSELAVLDGDSKESDESMAHDVASNGISGLELGLDLT |
| | | | LGLEPVSRAHHVVPVKKRRVEAYGSGDVDTCKMELRLE* |
| 13 | PtaLBD30 | eugene3.00140160 | MSTATTNPINTGAGGGSSGGGGGGGGGGGGGGGPCGACK |
| | polypeptide | | FLRRKCVPGCIFAPYFDSEQGAAHFAAVHKVFGASNVSK |
| | sequence | | LLLHIPVHKRLDAVVTICYEAQARLRDPVYGCVAHIFAL |
| | | | QQQVVNLQAELSYLQAHLAALEVPSPPPPPPPTLVTPPP |
| | | | LSIADLPSASSIPAAYDLSCLFDPMMQPSWSNMQPQRQM |
| | | | DIRQFGGSGGSSGTGGGDLQALARELLHRRGSPPPGFMS |
| | | | CSDTLASPSISK* |
| 14 | PtaLBD15 | eugene3.00131258 | MSRDRERSEELGKRIKRESDASSFMGRRQMLGPPVILNT |
| | polypeptide | | VTPCAACKLLRRRCAEECPFSPYFSPHEPQKFAAVHKVY |
| | sequence | | GASNVSKLLMEVPESQRVDTANSLVYEANLRLRDPVYGS |
| | | | MGAISALQQQIQSLQAELSAIRAEILNYKYREAAAATNI |
| | | | ISSTHPALVSSATVSISTPSQTLAPPPQPPPPSVVVSSS |
| | | | SSSSLYTPPTSTSGYSTISSENNVPYFD* |
| 15 | PtaLBD21 | fgenesh4_pg.C_LG | MRNQEPRASSSCAACKFLKRRCTPNCIFAPYFRSDEPKK |
| | polypeptide | _VIII000617 | FARVHKVFGASNVSKILIEVPEEQREDTVNSLAYEAEAR |
| | sequence | | LTDPVYGCIGAIALLQRKMVELQVDLAIARARLARYAAN |
| | | | SPPILNDHGSMIPTLAEFPACGGLVDSFNQSSSDTMNDF |
| | | | SQFPFIS* |
| 16 | PtaLBD18 | fgenesh4_pm.C_L | MSTTTTNPINSGVGGGSSGGGGAGGSSGGGGGPCGACKF |
| | polypeptide | G_II000686 | LRRKCVPGCIFAPYFDSEQGAAHFAAVHKVFGASNVSKL |
| | sequence | | LLHIPAHKRPDAVVTICYEAQARLRDPVYGCVAHIFALQ |
| | | | QQVVNLQAELSYLQAHLAAMEVPSPPPPPPPALVTSPPF |
| | | | SIADLPSASSIPGAAYDLSSLFDPMVQPSWSMQPRQLDP |
| | | | HQFGGSSGTSETSGGDLQALARELLHRRGSLPPGSVPCS |
| | | | DALASSSISK* |

### Example 4

### RT-PCR Analysis Shows PtaLBD15 and PtaLBD15 are Predominantly Expressed in the Xylem in Regions of the Stem Initiating Secondary Growth

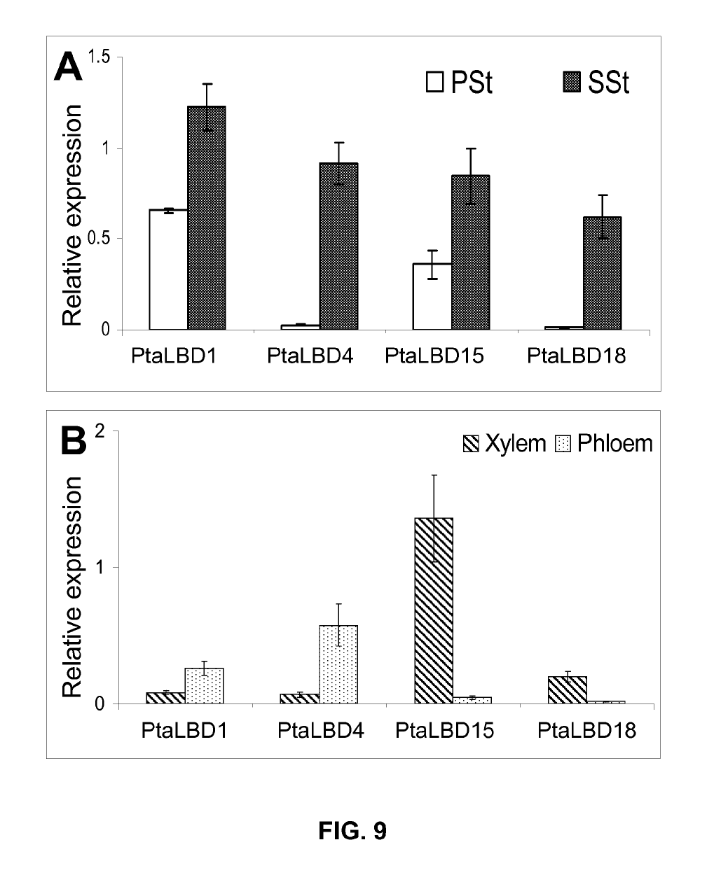

Four LBD genes, including the activation tagged PtaLBD1, were found to be differentially upregulated in stems undergoing secondary growth (Table 1). To validate these findings, expression analysis was performed using tissues derived from WT-717 poplar plants. The data show that poplar LBD genes are predominantly expressed in secondary phloem and xylem. All four genes demonstrated predominant expression in stems undergoing secondary growth (FIG. 9A). PtaLBD4 and PtaLBD18 were almost exclusively expressed in secondary woody stems (FIG. 9A). Additionally, PtaLBD1 and PtaLBD4 were mainly expressed in phloem, whereas PtaLBD 15 and PtaLBD 18 in xylem tissues (FIG. 9B).

### Example 5

### Generation of Transgenic Poplar Plants Expressing PtaLBD15

As for the recapitulation of the activation tagged gene, pK7WG2 will be used. The full length cDNA of PtaLB15 will be inserted between CaMV35S promoter (or a xylem-specific promoter) and a 35S terminator. Agrobacterium will be transformed with the construct using the freeze-and-thaw method and the transformation verified via PCR and restriction digest of the plasmid. The Agrobacterium-mediated transformation procedure will be used, as described previously (Filichkin, S. A. et al. (2006), Plant Cell Rep. 25: 660-667). Approximately 20 independent events will be regenerated. All plants that are rooted on transgenic media will be PCR-verified for the presence of the transgene before any molecular or phenotypic characterization. The expression will first be characterized to ensure that the transgene is highly expressed. All plants will be propagated in vitro and transferred to greenhouse conditions. All experiments will use WT control 717 plants and transgenic controls carrying the empty vectors. Each independent event will be represented by 15 ramets (clonal propagules). Complete randomized block design will be used to study growth of transgenic plants in the greenhouse. Plants will be grown for approximately 2 months in pots and detailed biometric measurements of leaf and stem development on weekly bases will be performed. At the end of the experiment, plants will be sampled for analysis of wood development (see below for more detail), as well as for determining total above and below ground biomass. ANOVA will be used to analyze biometric and biomass measurements and identify assess the significance of the differences between the various transgenic manipulations.

To analyze wood development we will collect stem sections in three parts of the stems corresponding approximately to primary, transition and secondary growth. Stem sections will be fixed in cold FAA and subsequently processed. First, the width of different tissues will be measure and the tissues will be inspected for abnormalities in their development. Easily distinguishable xylem, phloem and cambial zones will be measured at four different positions in the section. To analyze morphology of different cell types, FAA fixed stem sections will be macerated by boiling stem sections in H₂O₂:glacial acetic acid solution (1:1) for 6 h, dye with safranin and record images using a fluorescence microscope (E-400, Nikon, Tokyo) equipped with a digital imaging system. Measurement of length and width of vessels and fibers will be performed using ImageJ software. Changes in the pattern of vessel cell wall thickenings (pitted vs. spiral) and their frequency of occurrence will be recorded using fluorescence images under UV illumination.

Two types of cell divisions occur during secondary phloem and xylem development-anticlinal and periclinal. The anticlinal division result in addition of a new file of cambium initials to the vascular cambium to accommodate the growth in circumference and are restricted to the cambium. The periclinal division determines the number of xylem or phloem cells in each radial file. The strongest effect of LBDs is expected to be seen on the periclinal divisions because that is where the LBD genes are expected to be expressed and functioning. We will therefore assess changes in the frequency of periclinal divisions as previously described (Espinosa-Ruiz, A. et al. (2004), Plant Journal 38:603-615; Nilsson, J. et al. (2008), Plant Cell 20: 843-855; Schrader, J. et al. (2004), Plant Cell 16: 2278-2292). Stem sections will be subjected to overnight pectinase treatment that facilitates separation of developing secondary xylem/phloem. Bark will be then separated from the stem and developing secondary xylem or phloem cells including fusiform cambium derivatives gently lifted from the exposed xylem and bark sides respectively. The cells will be mounted, visualized using toluidine blue and approximately 500 cells inspected. Frequency of cells in mitotic phase will be recorded. the frequency of anticlinal division using series of cross sections will also be assessed as previously described (Schrader et al. 2004).

Substantial changes in the proportion of different cell and tissue types in the wood, and therefore changes in the overall wood physical and chemical properties are expected in the LBD transgenic plants. A significant increase in girth growth rate is expected. It is expected that this will be predominantly contributed by increased xylem production. Plants are also expected to reach harvest age much faster due to the increased girth growth. Because of the increased production of xylem, higher cellulose and/or lignin content per wood dry weight is expected. Because these changes can be of substantial interest basic analysis of wood physicochemical properties will be performed. The content of the two major components of the cell wall—lignin and cellulose will be determined. Before cellulose and lignin quantification the wood stem samples will be extracted with acetone. Cellulose content determination will be performed as previously described (Updegraff, D. M. (1969), Analytical Biochemistry 32:420-424). Homogenized stem wood will be first hydrolyzed in acetic nitric acid reagent for removing of all non cellulose sugars and lignin. The remaining cellulose will be treated with 67% sulfuric acid, then measured with anthrone reagent (Updegraff, D. M. (1969)). Lignin will be measured by modified acetyl bromide method (Yokoyama, T. et al. (2002), J. Agric. Food Chem. 50:1040-1044). using ferulic acid as a standard and UV absorbance at 280 nm. Measurements will be repeated with two sets of independently-grown plants and five ramets per independent event.

Several physical wood properties will also be determined. Green wood density (Den-g, g/cm³), basic wood density (Den-d, g/cm³), and moisture content (MC, %) will be measured of wood samples collected at the base of the stem. For each sample, the green disc mass and green volume, will be determined using water displacement. The stem will be kiln-dried at 105° C. and again weighed. Den-g and Den-d will be estimated for each tree as sample mass (g)/disc green volume (cm³). MC will be estimated as [(Den-g−Den-d)/Den-g]×100. We will also determine the modulus of elasticity (MOE).

For events that show changes in either chemical or physical wood properties the secondary cell wall structure using transmission electron microscopy will be studied in more detail. Approximately 1-2 mm² segments of dry wood will be extracted with cyclohexane-ethanol and embedded in Spurr's epoxy resin. Sixty to ninety nanometers thick sections will be cut with ultramicrotome and stained for 10 min with 1% KMnO₄.

### Example 6

### Generation of Transgenic Poplar Plants Expressing PtaLBD15

As for the recapitulation of the activation tagged gene, pK7WG2 will be used. The full length cDNA of PtaLB18 will be inserted between CaMV35S promoter (or a xylem-specific promoter) and a 35S terminator. Transformation and analysis of plants will be performed as for PtaLBD 15 in Example 5. Similar results, as described in Example 5 are expected.

1. An isolated polynucleotide comprising a contiguous coding sequence encoding a polypeptide having at least 95% identity to at least one amino acid sequence selected from SEQ ID NOs: 9-16.

2. An isolated polynucleotide comprising the complement of the polynucleotide of claim 1.

3. The isolated polynucleotide of claim 1, wherein the amino acid sequence is selected from SEQ ID NO: 14 and SEQ ID NO: 18.

4. A vector comprising the polynucleotide of claim 1.

5. A polynucleotide construct comprising a promoter operably linked to the polynucleotide of claim 1.

6. The polynucleotide construct of claim 5, wherein the promoter is a heterologous promoter.

7. A woody plant cell comprising the polynucleotide construct of claims 5.

8. A woody plant comprising the woody plant cell of claim 7.

9. The woody plant of claim 8, wherein the woody plant exhibits increased secondary growth relative to a control woody plant lacking the polynucleotide construct.

10. The woody plant of claim 8, wherein the woody plant exhibits increased phloem production.

11. The woody plant of claim 8, wherein the woody plant exhibits increased xylem production.

12. The woody plant of claim 8, wherein the woody plant exhibits increased internode number relative to a control woody plant lacking the polynucleotide.

13. The woody plant of claim 8, wherein the woody plant exhibits increased stem diameter relative to a control woody plant lacking the polynucleotide.

14. The woody plant of claim 8, wherein the woody plant is a tree.

15. The tree of claim 14, wherein the tree is a poplar, aspen, pine, eucalyptus or sweetgum tree.

16. An isolated polypeptide comprising an amino acid sequence having at least 95% identity to at least one amino acid sequence selected from SEQ ID NOs: 9-16.

17. A method of producing a transgenic woody plant comprising:
(a) introducing into a woody plant cell a polynucleotide encoding a polypeptide comprising an amino acid sequence having at least 95% identity to at least one amino acid sequence selected from SEQ ID NOs: 9-16; and
(b) regenerating the transformed cell to produce a transgenic woody plant.

18. The method of claim 17, wherein the transgenic woody plant exhibits increased secondary growth relative to a control woody plant lacking the polynucleotide.

19. The method of claim 17, wherein the transgenic woody plant exhibits increased phloem production.

20. The method of claim 17, wherein the transgenic woody plant exhibits increased xylem production.

21. The method of claim 17, wherein the transgenic woody plant exhibits increased internode number relative to a control woody plant lacking the polynucleotide.

22. The method of claim 17, wherein the transgenic woody plant exhibits increased stem diameter relative to a control woody plant lacking the polynucleotide.

23. The method of claim 17, wherein the transgenic woody plant is a transgenic tree.

24. The method of claim 23, wherein the transgenic tree is a poplar, aspen, pine, eucalyptus or sweetgum tree.

25. A transgenic woody plant produced by the method of claim 17.

26. A method of producing a transgenic woody plant comprising:
(a) introducing into a woody plant cell a polynucleotide comprising a sequence having at least 95% identity to the complement of at least one of SEQ ID NOs: 1-8; and
(b) regenerating the transformed cell to produce a transgenic woody plant.

27. The method of claim 26, wherein the transgenic woody plant exhibits decreased secondary growth.

28. A method of producing a transgenic woody plant comprising:
(a) introducing into a woody plant cell a polynucleotide encoding a polypeptide selected from LBD11, LBD41, LBD4, LBD30, LBD15, LBD21, and LBD18; and
(b) regenerating the transformed cell to produce a transgenic woody plant.

29. The method of claim 28, wherein the transgenic woody plant exhibits increased secondary growth relative to a control woody plant lacking the polynucleotide.

30. The method of claim 28, wherein the transgenic woody plant exhibits increased phloem production.

31. The method of claim 28, wherein the transgenic woody plant exhibits increased xylem production.

32. The method of claim 28, wherein the transgenic woody plant exhibits increased internode number relative to a control woody plant lacking the polynucleotide.

33. The method of claim 28, wherein the transgenic woody plant exhibits increased stem diameter relative to a control woody plant lacking the polynucleotide.

34. The method of claim 28, wherein the transgenic woody plant is a transgenic tree.

35. The method of claim 34, wherein the transgenic tree is a poplar, aspen, pine, eucalyptus or sweetgum tree.

36. The method of claim 28 wherein the polypeptide is selected from PtaLBD11, PtaLBD41a, PtaLBD4, PtaLBD41b, PtaLBD30, PtaLBD15, PtaLBD21, and PtaLBD18.

37. A transgenic woody plant produced by the method of claim 28.
